(19) United States
(12) Patent Application Publication
(10) Pub. No.: US 2008/0154153 A1
(43) Pub. Date: Jun. 26, 2008

(54) MULTIPLE-WIRE SYSTEMS AND METHODS FOR ABLATION OF OCCLUSIONS WITHIN BLOOD VESSELS

(76) Inventor: Richard R. Heuser, Phoenix, AZ (US)

Correspondence Address: KOLISCH HARTWELL, P.C., 520 SW YAMHILL STREET, Suite 200, PORTLAND, OR, 97204, US

(21) Appl. No.: 12/043,675

(22) Filed: Mar. 6, 2008

Related U.S. Application Data
(63) Continuation-in-part of application No. 10/927,340, filed on Aug. 25, 2004.

(51) Int. Cl. A61M 25/09

(52) U.S. Cl. 600/585

(57) ABSTRACT

Multiple-wire systems for the ablation of occlusions within blood vessels. Systems include two or more concentric wires configured for percutaneous insertion in a blood vessel, the wires configured to ablate an occlusion within the blood vessel.

### RELATED APPLICATION

This application is a continuation-in-part of, and claims benefit under 35 U.S.C. § 120 to, U.S. patent application Ser. No. 10/927,340, entitled “CATHETER GUIDEWIRE SYSTEM USING CONCENTRIC WIRES,” filed on Aug. 25, 2004, the content of which is hereby incorporated by reference for all purposes.

### BACKGROUND

The present disclosure relates to multiple-wire systems for ablation of occlusions within blood vessels, the systems adapted to maneuver through bends, bifurcations, narrowing vessels, and other complications within blood vessels.

### SUMMARY

Multiple-wire systems for the ablation of occlusions within blood vessels according to the present disclosure may include two or more concentric wires configured for percutaneous insertion in a blood vessel. Some embodiments further include a radio-frequency device configured to deliver radio-frequency energy to one or more of the concentric wires. Some embodiments include one or more concentric wires having a textured outer surface that aids in the passage of the wire through a blood vessel.

Methods according to the present disclosure for ablating an occlusion within a blood vessel may include percutaneously inserting two or more concentric wires into a blood vessel, feeding the concentric wires through the blood vessels, manipulating one or more of the concentric wires to engage the occlusion, and applying radio-frequency energy to a distal end of one or more of the concentric wires to ablate the occlusion.

### BRIEF DESCRIPTION OF THE DRAWINGS

FIG. 1 is a schematic illustration of multiple-wire systems according to the present disclosure that include two concentric wires.

FIG. 2 is a schematic cross-sectional illustration of the systems of FIG. 1 taken along line 2-2.

FIG. 3 is a schematic illustration of multiple-wire systems according to the present disclosure that include three concentric wires.

FIG. 4 is a schematic cross-sectional illustration of the systems of FIG. 3 taken along line 4-4.

FIG. 5 is a schematic cross-sectional illustration of a wire tip of a multiple-wire system according to the present disclosure, the wire tip including a portion of a radio-frequency device therein.

FIG. 6 is a schematic cross-sectional illustration of a wire tip of a multiple-wire system according to the present disclosure, the wire tip having a mandril extending therethrough and a portion of a radio-frequency device mounted on the distal end of the mandril.

FIG. 7 is a schematic cross-sectional illustration of a portion of a wire of a multiple-wire system according to the present disclosure, the wire including an insulative coating and an exposed tip.

FIG. 8 is an isometric side view of a portion of a multiple-wire system according to the present disclosure, the system including wires that include a plurality of wound wire strands.

FIG. 9 is an isometric side view of a wire according to the present disclosure, the wire tapered and including a plurality of wound wire strands.

FIG. 10 is a cross-sectional side view of a multiple-wire system according to the present disclosure, the system including two concentric wires, each wire including a handle.

Fig. is a cross-sectional side view of a multiple-wire system according to the present disclosure, the system including three concentric wires, each wire including a handle.

FIGS. 12A and 12B are cross-sectional views of two wires of a multiple-wire system according to the present disclosure, the wires extending around a bend adjacent a bifurcation in a human blood vessel, showing the difference in performance between a transitionless wire (12A) and a wire with a transition (12B).

FIG. 13 is a cross-sectional view, from a perspective of facing the patient, of contralateral access by a multiple-wire system according to the present disclosure from the right iliac artery to the left iliac artery.

FIG. 14 is a cross-sectional view of a two-wire system according to the present disclosure being maneuvered into a branch of a blood vessel.

FIG. 15 is a cross-sectional view of a two-wire system according to the present disclosure with a catheter being maneuvered into a branch of a blood vessel.

### DETAILED DESCRIPTION

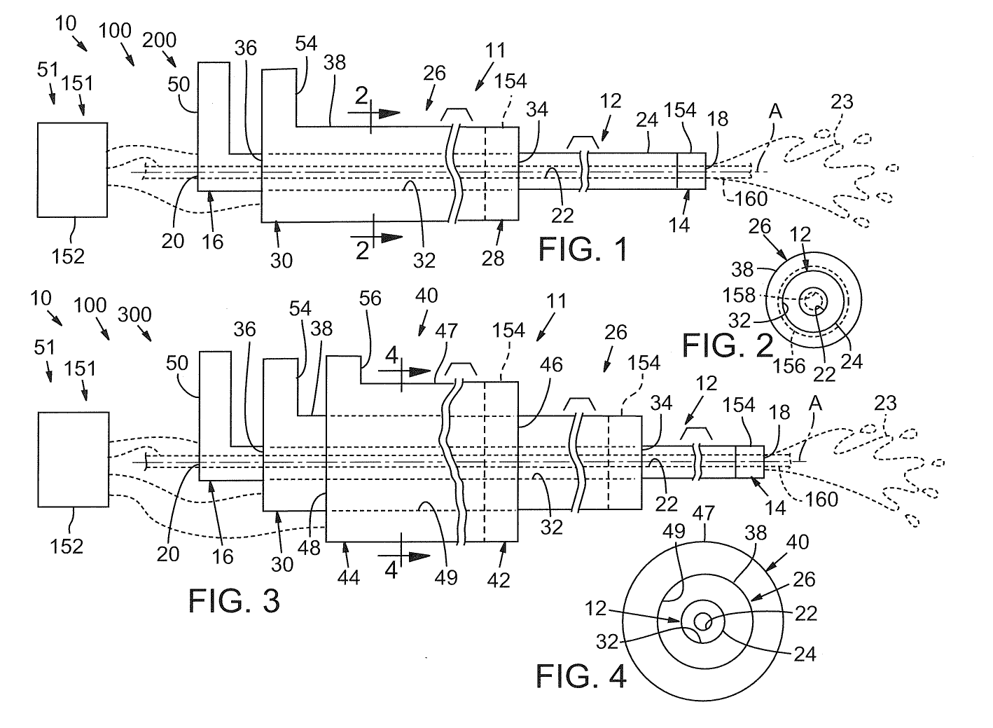

Multiple-wire systems according to the present disclosure are schematically illustrated in FIGS. 1-4 and are generally indicated at 10. Systems 10 may include two or more concentric wires 11. Wires 11 may additionally or alternatively be described as guidewires. Wires 11 may (but are not required to) be constructed at least partially with a hydrophilic material (e.g., coated with a polytetrafluoroethylene (PTFE) or plastic covering) selected for a particular procedure being performed. As used herein, the term ‘hydrophilic’ refers to a property of a material where the material becomes slippery when subjected to a fluid, such as a liquid. Accordingly, wires 11 made of a hydrophilic material may be less likely to snag within a blood vessel or to accidentally poke through the wall of a blood vessel, such as when the wire is being routed around a bend or turn of a vessel.

Multiple wire systems according to the present disclosure may (but are not required to) further include a treatment device 51 in the form of a radio-frequency device 151 operatively connected to one or more of wires 11. Such embodiments that include a radio-frequency device 151 may be referred to as radio-frequency, or RF, wire systems 100. Additionally or alternatively, systems 10 according to the present disclosure may include a treatment device 51 that incorporates a laser energy device, an optical coherent reflectometry (OCR) device, an ultrasound device, or any other device suitable for mounting on a wire or catheter and for controlling from outside the body while inserted in the body.

A wire 11 according to the present disclosure may have a generally cylindrical outer surface that defines an outer diameter. Additionally or alternatively, a wire 11 may have an outer surface that generally tapers at least partially along its overall length. For example, a wire may have a greater diameter at its proximal end than at its distal end. Such a wire may have a proximal end diameter equal to about 0.024 inches and a distal end diameter equal to about 0.01 inches; however, other configurations are equally within the scope of the present disclosure. Additionally or alternatively, a wire may have a generally cylindrical outer surface for a portion of its length and a tapered outer surface for another portion of its length. Other configurations are equally within the scope of the present disclosure, and the schematic illustrations of FIGS. 1-4 are not to be interpreted as limiting wires 11 to having only cylindrical outer surfaces with constant outer diameters.

An example of a two-wire system is schematically illustrated in FIGS. 1 and 2, and an example of a three-wire system is schematically illustrated in FIGS. 3 and 4. Systems with more than three wires are equally within the scope of the present disclosure.

In the non-exclusive example illustrated in FIG. 1, an RF system 100 including two concentric wires is generally indicated at 200. System 200 includes a first, inner wire 12, a second wire 26 through which first wire 12 extends, and a radio-frequency device 151 operatively connected to one or both of the first and second wires.

First wire 12 includes a distal end 14 and a proximal end 16, and has a length that may be selected for a particular type of procedure to be conducted in a human blood vessel. For example, first wire 12 may be between about 150 cm and about 300 cm; however, other lengths are equally within the scope of the present disclosure. Inner wire 12 may (but is not required to) include an opening 18 adjacent distal end 14, an opening 20 adjacent proximal end 16, and a central lumen 22 extending between the proximal and distal openings, which may define an inner diameter of first wire 12.

Embodiments that include a lumen 22 within first wire 12 may be used to deliver a fluid 23 (including liquids and gases), such as (but not limited to) water, saline, compressed air or other gas, pharmaceuticals (whether in liquid or gas form), etc., to a site within a blood vessel. For example, a delivered fluid may be used to break up an occlusion and/or to expand an already partially open occlusion. In such embodiments, lumen 22 may (but is not required to) be coated with a fluid-impervious coating to prevent the migration of fluid 23 through the wall of first wire 12 when a system is used to deliver a fluid to a site within a blood vessel.

First wire 12 may (but is not required to) have a generally cylindrical outer surface 24 defining an outer diameter, which may be between about 0.004 and about 0.014 inches, or may be larger or smaller as selected for a particular procedure and for compatibility with other wires, catheters, sheaths, and other equipment. Alternatively, as mentioned, first wire 12 may have an outer surface 24 that tapers at least partially along its length.

Additionally or alternatively, first wire 12 may include an outer surface 24 that has a textured surface that is configured to provide a mechanism for aiding with the insertion and/or passage of wire 12 through a blood vessel in response to an operator manipulating the wire (e.g., by twisting it), as discussed in more detail below in reference to FIG. 8.

Inner wire 12 may be provided with a handle portion 50 adjacent proximal end 16 that a user (e.g., a physician) may use in manipulating the wire about and along a central axis A of the wire. Handle portion 50 may (but is not required to) be removable in some embodiments. Wire 12 may be constructed with a hydrophilic material (e.g., coated with a Teflon or plastic covering) selected for a particular procedure being performed.

Second wire 26 may be constructed to be deployed over first wire 12. Additionally or alternatively, second wire 26 may be described as being constructed to receive first wire 12, such that first wire 12 is deployed within second wire 26 after second wire 26 has already been deployed within a blood vessel.

Second wire 26 includes a distal end 28 and a proximal end 30, and has a length that is compatible with first wire 12, for example, less than the length of first wire 12. The length of second wire 26 may be selected for a particular type of procedure to be conducted in a human blood vessel. For example, the length may be between about 125 cm and about 275 cm; however, other lengths are equally within the scope of the present disclosure. Second wire 26 includes an opening 34 adjacent distal end 28, an opening 36 adjacent proximal end 30, and a central lumen 32 extending between the distal and proximal openings, which may define an inner diameter of second wire 26. The inner diameter is sized so as to receive and permit first wire 12 to extend therethrough and permit relative movement between the first and second wires. In some embodiments, though not required, central lumen 32 of second wire 26 may be configured to compliment the outer surface 24 of inner first wire 12. For example, in embodiments where the outer surface of the first inner wire is configured with a textured surface, the central lumen of the second wire may (but is not required to) be configured with a corresponding textured inner surface that is adapted to compliment the textured outer surface of the inner wire, as discussed in more detail below in reference to FIG. 8.

Second wire 26 may have a generally cylindrical outer surface 38 defining an outer diameter, which may be between about 0.008 and about 0.035 inches, or may be larger or smaller as selected for a particular procedure and for compatibility with other wires, catheters, sheaths, and other equipment. Alternatively, as mentioned, second wire 26 may have an outer surface 38 that tapers at least partially along its length.

Additionally or alternatively, second wire 26 may include a textured outer surface that is configured to provide a mechanism for aiding with the insertion and/or passage of wire 26 through a blood vessel in response to an operator manipulating the wire (e.g., by twisting it), as discussed in more detail below in reference to FIG. 8.

Second wire 26 may be provided with a handle portion 54 adjacent proximal end 30 that a physician may use in manipulating the wire about and along central axis A. Handle portion 54 may (but is not required to) be removable in some embodiments.

Second wire 26 may (but is not required to) have a rigidity selected to be greater than that of first wire 12, thus providing the system with an overall variable rigidity which depends on the extent to which the first wire extends out of the second wire.

One or more of wires 11 may (but are not required to) be constructed in sections. In such embodiments the wire(s) may be constructed without transitions between the sections. For example, wires 11 may be used in crossing a bifurcation in the blood vessel, and may be provided with a rigidity selected to allow the bifurcation crossing. Rigidity may be controlled by the use of braiding or the selection of various materials. For example, nitinol is flexible, but it becomes stiffer as more stainless steel is added.

As mentioned, RF wire systems 100 may include an RF device 151. Such devices may include an RF-generating device 152 operatively connected to one or more of wires 11 and configured to generate radio-frequency energy that may be delivered to at least a portion of one or more of wires 11. For example, as illustrated in FIG. 1, RF-generating device 152 may deliver RF energy to first wire 12. Additionally or alternatively, RF-generating device 152 may deliver RF energy to second wire 26. Examples of RF-generating devices and RF devices in general are disclosed in U.S. Pat. Nos. 6,190,379, 6,485,489, and 7,229,469, and U.S. patent application Ser. Nos. 11/433,198 and 11/688,785, the contents of which are hereby incorporated by reference for all purposes. Additional examples of RF-generating devices and RF devices in general include the Boa System™, the Boa-Surg Device™, and the Boa-Cathe Device™ offered by QuantumCor, Inc. of San Clemente, Calif. and the Safe-Cross® RF Crossing Wire device offered by IntraLuminal Therapeutics, Inc. of Carlsbad, Calif. Such devices may be used with, or adapted for use with, systems 100 according to the present disclosure to provide the RF energy that may be delivered to one or more of wires 11.

In some embodiments of RF wire systems 100, one or more of wires 11 may include an RF-delivery tip 154 positioned at the distal end thereof. Such delivery tips may be configured to receive RF energy from RF-generating device 152 and deliver the RF energy to an occlusion, for example, or other blockage or structure to be ablated during a procedure. Various configurations of delivery tips are within the scope of the present disclosure and are discussed in more detail below. Delivery tips 154 may be described as comprising a portion of an RF device 151.

The RF energy may be routed to the RF delivery tip(s) in any number of ways that may be appropriate for a specific configuration of system 100. For example, the bulk of a wire 11 (e.g., the conductive portions thereof) may be charged with the RF energy. Alternatively, the RF energy may be delivered solely to a tip portion via a wire or other structure that is generally insulated from the bulk of the wire 11 to which a specific tip 154 is being charged.

In embodiments where the bulk of a wire 11 is charged with RF energy, the given wire may be coated with an insulative covering to avoid transfer of the RF energy to an adjacent concentric wire 11, for example, as schematically illustrated in FIG. 2 at 156 as coating the outer surface 24 of first wire 12. Additionally or alternatively, the lumen of a given wire 11 may be coated with an insulative lining, for example, as schematically illustrated in FIG. 2 at 158 as coating the central lumen 22 of first wire 12. Other configurations are equally within the scope of the present disclosure. A non-exclusive example of an insulative material that may be used in such embodiment is polytetrafluoroethylene (PTFE).

In embodiments where first wire 12 includes a central lumen 22, systems 10 according to the present disclosure may (but are not required to) further include a mandril 160 configured to extend through first wire 12. Mandril 160 may be considered a wire 11 according to the present disclosure; however, mandril 160 may or may not include a central lumen, and may generally be a cylindrical wire adapted to provide treatment or diagnostic abilities for systems 10. For example, mandril 160 may be charged with RF energy for the ablation of an occlusion or other tissue. Examples of mandrils 160 are disclosed in U.S. Pat. No. 6,190,379, incorporated above.

FIGS. 3 and 4 schematically illustrate an example of a three-wire RF wire system 300. Like systems 200, systems 300 include a first wire 12, a second wire 26 through which first wire 12 extends, and a radio-frequency device 151. A third wire 40 is also provided that may be constructed to be deployed over second wire 26. Additionally or alternatively, third wire 40 may be described as being constructed to receive second wire 26, such that second wire 26 is deployed within third wire 40 after third wire 40 has already been deployed within a blood vessel.

Third wire 40 includes a distal end 42 and a proximal end 44, and has a length that is compatible with first and second wires 12, 26, for example less than that of second wire 26. The length of third wire 40 may be selected for a particular type of procedure to be conducted in a human blood vessel. For example, the length may be between about 100 cm and 250 cm; however, other lengths are equally within the scope of the present disclosure. Third wire 40 includes an opening 46 adjacent distal end 42, an opening 48 adjacent proximal end 44, and a central lumen 49 extending between the proximal and distal openings, which may define an inner diameter of third wire 40. The inner diameter is sized so as to receive and permit second wire 26 to extend therethrough and permit relative movement between the second and third wires. In some embodiments, though not required, central lumen 49 of third wire 40 may be configured to compliment the outer surface 38 of second wire 26. For example, in embodiments where the outer surface of the second wire is configured with a textured surface, the central lumen of the third wire may (but is not required to) be configured with a corresponding textured inner surface that is adapted to compliment the textured outer surface of the second wire, as discussed in more detail below in reference to FIG. 8.

Third wire 40 may have a generally cylindrical outer surface 47 defining an outer diameter, which may be between about 0.010 and about 0.035 inches, or may be larger or smaller as selected for a particular procedure and for compatibility with other wires, catheters, sheaths, and other equipment. Alternatively, as mentioned, third wire 50 may have an outer surface 47 that tapers along at least a portion of its length.

Additionally or alternatively, third wire 40 may include a textured outer surface that is configured to provide a mechanism for aiding with the insertion and/or passage of third wire 40 through a blood vessel in response to a user manipulating the wire (e.g., by twisting it), as discussed in more detail below in reference to FIG. 8.

Third wire 40 may be provided with a handle portion 56 adjacent proximal end 44 that a physician may use in manipulating the wire about and along central axis A. Handle portion 56 may (but is not required to) be removable in some embodiments.

Third wire 40 may (but is not required to) have a rigidity selected to be greater than that of the first and second wires, thus providing the system with an overall variable rigidity that depends on the extent to which the second wire extends out of the third wire, and the extend to which the first wire extends out of the second wire.

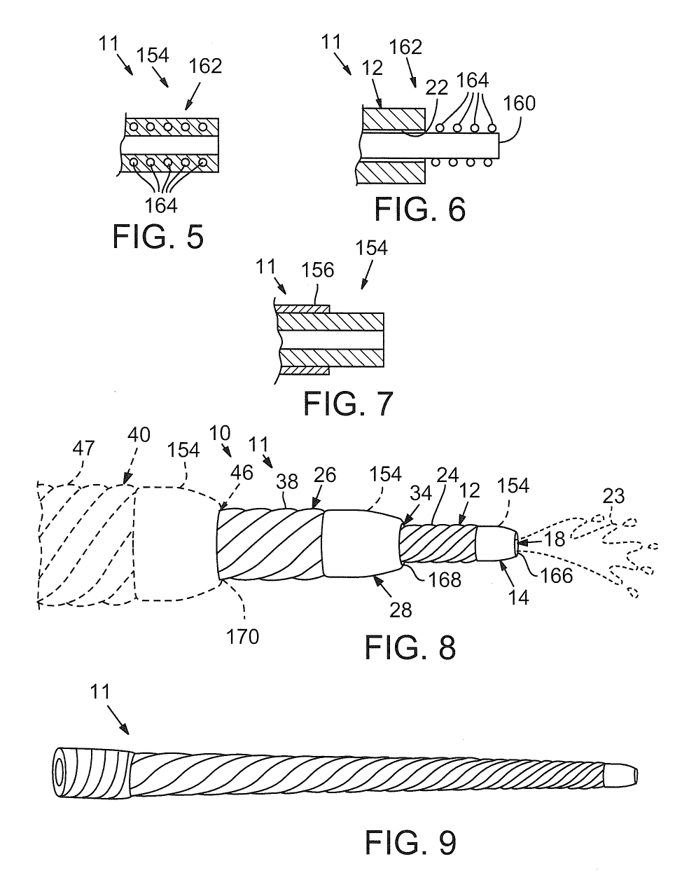

As mentioned, one or more of wires 11 may include an RF-delivery tip 154 positioned at the distal end thereof. Non-exclusive examples of delivery tips 154 are illustrated in FIGS. 5-7. In the embodiment illustrated in FIG. 5, wire 11 includes an RF energy collection structure 162 in the form of a plurality of spaced apart rings 164 embedded within tip 154. Additionally or alternatively, collection structure 162 may take the form of a coil. Rings 164, or similarly a coil, may be configured to absorb RF energy delivered to tip 154 by an associated RF-generating device through the bulk of wire 11 or through an associated wire or other structure that is generally insulated from the bulk of wire 11. A non-exclusive example of a material that may be appropriate for the construction of rings 164, or a coil, is gold. Other configurations of RF energy collection and delivery structure are equally within the scope of the present disclosure and may be incorporated into systems 10.

FIG. 6 illustrates a non-exclusive example of a system 10 that includes a mandril 160 extending through a first wire 12. In such an embodiment, rather than energizing the distal end of the first wire 12 with radio-frequency energy, the distal end of the mandril may include radio-frequency energy collection structure 162 in the form of a plurality of spaced apart rings 164, or in the form of a coil, wrapped around the distal end of the mandril.

FIG. 7 illustrates yet another non-exclusive example of a delivery tip 154. In this embodiment, rather than including RF energy collection structure, the wire 11 includes an insulative covering 156 as discussed above, with the covering leaving at least a portion of the distal end of wire 11 exposed. In such an embodiment, the entire wire 11, or at least the bulk of wire 11, may be charged with RF energy, while only the distal end is exposed within a blood vessel when being used, and therefore may be used to ablate an occlusion or other tissue therein.

As discussed above, wires 11 of systems 10 may (but are not required to) include textured outer surfaces configured to provide a mechanism for aiding with the insertion of a wire through a blood vessel, or occlusion therein, in response to an operator manipulating the wire. For example, one or more of the outer surfaces may have a spiraled, screw-like, or threaded configuration that aids with the insertion and/or passage of a wire through a blood vessel in response to an operator twisting the wire 11. For example, a wire 11 may be formed by a plurality of wound or braided wires or wire strands. A non-exclusive example of a system 10 including such structure is illustrated in FIG. 8.

In the illustrated non-exclusive embodiment of FIG. 8, a system 10 includes a first wire 12 and a second wire 26, constructed of a plurality of smaller wires wound about their respective lumens. Accordingly, outer surface 24 of first wire 12 and outer surface 38 of second wire 26 provide a screw-like or threaded configuration. As illustrated in dashed lines, systems 10 having wires 11 with textured outer surfaces are not limited to two-wire systems, and may further include a third wire 40. Additional wires beyond three are equally within the scope of the present disclosure.

Additionally, though not required, in embodiments where an outer wire (i.e., a second, third, or further wire having a concentric wire extending therethrough) includes a textured configuration as discussed, the central lumen of the outer wire may be configured with a corresponding textured inner surface (e.g., in the form of female threads, or the like) that is adapted to compliment the textured outer surface of the inner wire, and thereby provide for relative screw-like displacement of the inner wire within the outer wire when a user twists the inner wire within the outer wire.

Multiple-wire systems having wires 11 with textured outer surfaces and an inner wire with a central lumen may be particularly well suited for the delivery of a fluid to a site within a blood vessel. By having a plurality of concentric wires, the fluid may be generally prevented from leaking through the structure that defines the textured outer surface. For example, in embodiments where the textured outer surface of a wire is defined by a plurality of wound or braided wire strands, depending on the fluid being used for a particular procedure, the fluid may tend to leak through the wound or braided wire strands. By having one or more outer wires, fluid that manages to leak through the structure of the inner wire may generally be contained by the outer one or more wires. Additionally or alternatively, the central lumen of the inner wire may be lined with a coating (e.g., potytetrafluoroethylene (PTFE), plastic, or other suitable material) to generally prevent migration of the fluid through the wall of the inner wire. Additionally or alternatively, a tube (e.g., constructed of PTFE, plastic, or other suitable material) may be positioned within the central lumen of one or more of the concentric wires.

Additionally, though not required, embodiments that include wires with textured surfaces may include RF-delivery tips 154 for the delivery of RF energy within a blood vessel.

Additionally or alternatively, in some embodiments the distal openings to wires 11 may be defined by a cutting edge configured to aid in the passage of wires 11 through blood vessels and through occlusions, other blockages, or tissue therein. For example, in the non-exclusive embodiment illustrated in FIG. 8, distal opening 18 of first wire 12 may be defined by a cutting edge 166, distal opening 34 of second wire 26 may be defined by a cutting edge 168, and distal opening 46 of third wire 40 may be defined by a cutting edge 170. Such cutting edges may simply be thinner than the thickness of the rest of the wire 11, or alternatively may be defined by serrations or other structure configured to aid in the cutting through an occlusion, blockage, or other tissue. Such configurations may be particularly useful in embodiments that include a textured outer surface of a wire 11, as discussed above. Accordingly, as a user inserts a wire 11 through a blood vessel and reaches an occlusion or other blockage within a blood vessel, the user may twist the wire causing the distal opening to cut through the blockage and thereby facilitate further insertion of the wire, with or without the addition of RF energy.

Wires 11 according to the present disclosure are not limited to being cylindrical, and as discussed above and illustrated in FIG. 9, may have portions that are generally tapered or cone-shaped. The non-exclusive example illustrated in FIG. 9 further includes a textured outer surface, although other configurations of tapered wires, including wire having smooth outer surfaces, are equally within the scope of the present disclosure.

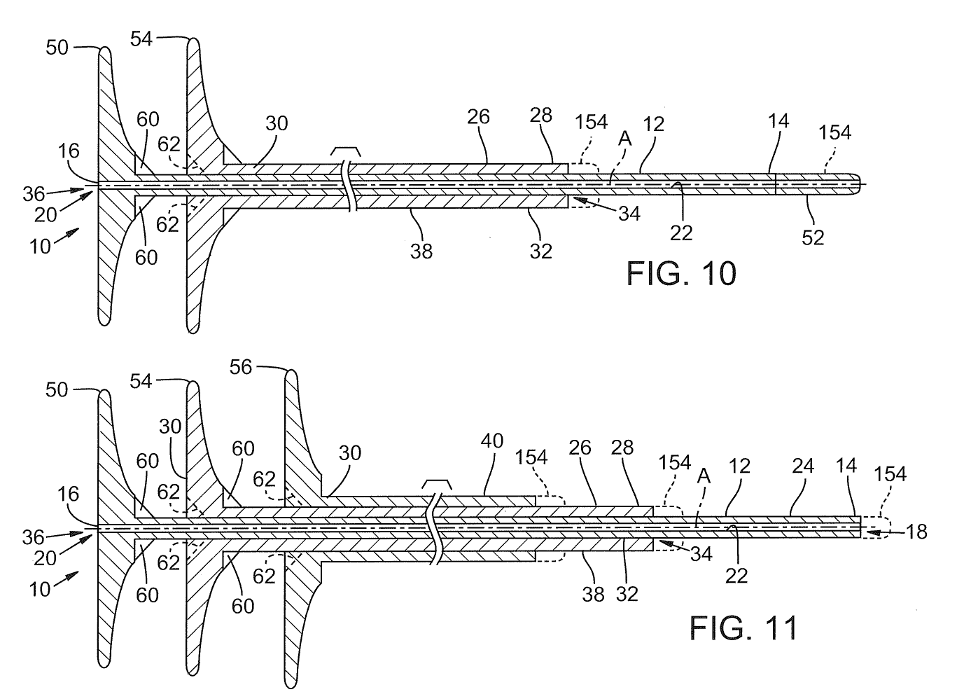

Further non-exclusive examples of systems 10 according to the present disclosure may be described as below in reference to FIGS. 10 and 11.

As shown in FIGS. 10 and 11, embodiments of guidewire systems of the present disclosure are multiple-wire systems indicated generally at 10. Systems 10 may include an inner wire 12 having a distal end 14 and a proximal end 16. Inner wire 12 has a length that may be selected for a particular type of procedure to be conducted in a human blood vessel, e.g., between about 180 cm and about 300 cm. Inner wire 12 may include an opening 18 adjacent distal end 14 and an opening 20 adjacent proximal end 16, and a central lumen 22 extending between the proximal and distal openings. Central lumen 22 defines an inner diameter for wire 12, and wire 12 also has a generally cylindrical outer surface 24 defining an outer diameter. The outer diameter of inner wire 12 may be between about 0.004 and about 0.014 inches, and may be any size therebetween, or larger or smaller as selected for the desired procedure and for compatibility with other wires, catheters, sheaths, and other equipment.

Inner wire 12 may be provided with a handle 50, preferably (but not required to be) removable, adjacent proximal end 16 that a physician may use in manipulating the wire about and along a central axis A of the wire. Wire 12 is may be constructed with a hydrophilic material selected for the particular procedure. For example, coating with a polytetrafluoroethylene (PTFE) or plastic covering makes a wire hydrophilic.

Wire 12 may be constructed without transitions between sections, if it includes any sections, of the wire. Inner wire 12 may be used in crossing a bifurcation in a blood vessel, and may be provided with a rigidity selected to allow the bifurcation crossing. Rigidity may be controlled by the use of braiding or the selection of various materials. For example, nitinol is flexible, but it becomes stiffer as more stainless steel is added.

As best seen in FIG. 10, inner wire 12 may optionally include a treatment or a diagnostic device 52 (e.g., in the form of an RF-delivery tip 154 as discussed above), typically located at the distal end 14 of wire 12. Alternatively, device 52 may be located in a more proximal position on wire 12, or may be located on the other wires or catheter to be described below. Device 52 may be any type of device useful for treating or diagnosing conditions in blood vessels, such as a radio-frequency energy device, a laser energy device, an optical coherent reflectometry (OCR) device, an ultrasound device, or any other device suitable for mounting on a wire or catheter and for controlling from outside the body while inserted in the body.

A second wire 26, preferably constructed to be deployed over inner wire 12, includes a distal end 28 and a proximal end 30 and a length preferably selected to be compatible with inner wire 12. A central lumen 32 of wire 26 extends between a distal opening 34 and a proximal opening 36. As mentioned, second wire 26 may include an RF-delivery tip 154 at its distal end.

Central lumen 32 of second wire 26 defines an inner diameter for the wire. Wire 26 may have a generally cylindrical outer surface 38 defining an outer diameter. The outer diameter of wire 26 may be between about 0.008 and about 0.035 inches, and may be any size therebetween, or larger or smaller as selected for the desired procedure and for compatibility with other wires, catheters, sheaths, and other equipment.

Wire 26 may be provided with a handle 54, preferably (but not required to be) removable, adjacent proximal end 30 that the physician may use in manipulating the wire about and along a central axis A of the wire. Second wire 26 may have a rigidity selected to be greater than that of inner wire 12, thus providing the system with an overall variable rigidity which depends on the extent to which the inner wire extends out of the second wire.

System 10 may also include a third or outer wire 40, as shown in FIG. 11, having proximal and distal ends with openings and a central lumen communicating therebetween, inner and outer diameters, and a generally cylindrical outer surface as for the other wires. Preferably third wire 40 is sized to fit over the second wire and includes a handle 56, preferably (but not required to be) removable, coupled adjacent the proximal end for manipulation of the third wire about and along central axis A. Third wire 40 may have a rigidity selected to be greater than the rigidity of the first wire and greater than the rigidity of the second wire, thus providing the system with an overall variable rigidity which depends on the extent to which the inner wire extends out of the second wire, and the extent to which the second wire extends out of the third wire. Third wire 40 may also include an RF-delivery tip 154 at its distal end.

Third wire 40 may have an outer diameter between about 0.010-inches and about 0.035-inches, and may be any size therebetween, or larger or smaller as selected for the desired procedure and for compatibility with other wires, catheters, sheaths, and other equipment. Typically, the length of the third wire is less than the length of the second wire, and the length of the second wire is less than that of the inner wire.

The multiple guidewire system may be combined with a catheter, such as catheter 58 that can be inserted over the wires, as shown in FIG. 13. Such a catheter may include a balloon and a stent placement apparatus. As described above, the catheter or one or more of the wires may be provided with a radio-frequency energy device, a laser energy device, and/or an optical reflectometry device for applying treatment within the blood vessel, or with other devices, including diagnostic devices such as ultrasound.

When the first, second, and third wires are coupled together, any of the handles of the first, second, and third wires may be used to manipulate all three wires, and also the wires may be manipulated relative to one another by simultaneous use of two or three of the handles. For example, as shown in FIGS. 10 and 11, handles 50 and 54 may include one or more forward-facing wings 60, which interlock with corresponding notches 62 in handles 54 and 56, when the handles are pushed together. When the wings and notches interlock, rotational movement of one handle will also rotate the wire attached to the interlocked handle. Alternatively, any other type of selective interlocking may be used, or the friction between the wires may provide for simultaneous movement, unless the handles are separately manipulated.

The length of the first wire may be between about 180 cm and about 300 cm, but may be other sizes as desired for particular procedures. The length of the second wire may be about 5 cm less than the first wire, and the length of the third wire may be about 5 cm less than the second wire.

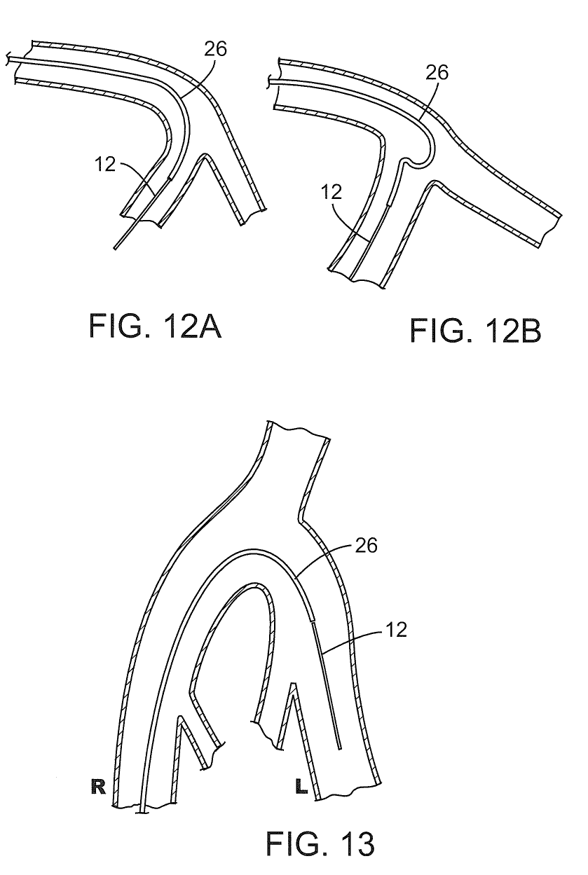

FIGS. 12A and 12B show two examples of a two-guidewire system, including inner wire 12 and outer wire 26, being used to extend around a bend and into one channel at a bifurcation in a human blood vessel. FIG. 12A shows the performance of a transitionless wire, which can extend around the corner without doubling over, while FIG. 12B shows the performance of a wire with a transition, which tends to double over. The transition typically occurs where two materials that are different in hydrophilicity or stiffness are directly joined, and a transitionless wire is typically provided by gradually changing the hydrophilicity or stiffness, or by other methods of preventing the abrupt transition.

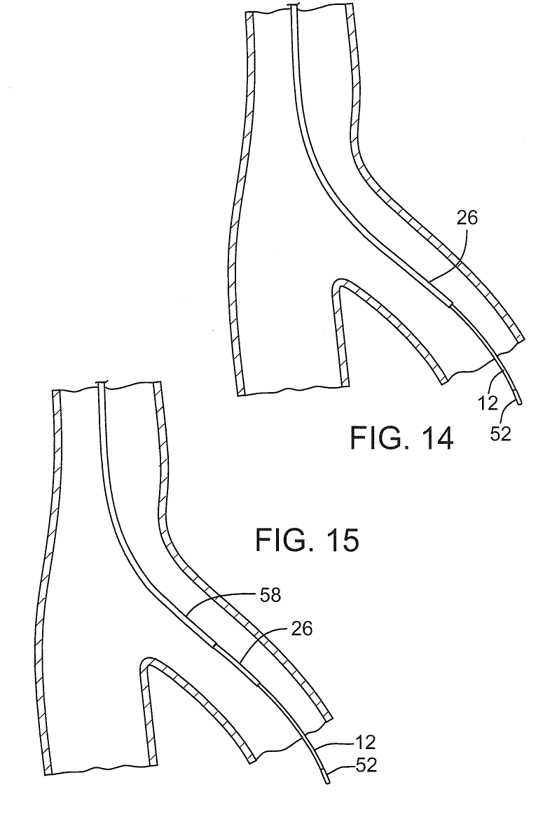

FIG. 13 shows contralateral access by the guidewire system from the right iliac artery R to the left iliac artery L. FIG. 14 shows a two-wire guidewire system, including inner wire 12 and outer wire 26, and treatment/diagnostic device 52, being maneuvered into a branch of a blood vessel. FIG. 15 shows the two-wire guidewire system with catheter 58 being maneuvered into a branch of a blood vessel.

The disclosure set forth above encompasses multiple distinct inventions with independent utility. While each of these inventions has been disclosed in a preferred form or method, the specific alternatives, embodiments, and/or methods thereof as disclosed and illustrated herein are not to be considered in a limiting sense, as numerous variations are possible. The present disclosure includes all novel and non-obvious combinations and subcombinations of the various elements, features, functions, properties, methods and/or steps disclosed herein. Similarly, where any disclosure above or claim below recites “a” or “a first” element, step of a method, or the equivalent thereof, such disclosure or claim should be understood to include one or more such elements or steps, neither requiring nor excluding two or more such elements or steps.

Inventions embodied in various combinations and subcombinations of features, functions, elements, properties, steps and/or methods may be claimed through presentation of new claims in a related application. Such new claims, whether they are directed to a different invention or directed to the same invention, whether different, broader, narrower, or equal in scope to the original claims, are also regarded as included within the subject matter of the present disclosure.

1. A multiple-wire system for ablation of an occlusion within a blood vessel, comprising:
an outer wire configured for percutaneous insertion in the blood vessel, the outer wire having a proximal end, a distal end, and a generally cylindrical outer surface, wherein the outer wire includes an opening adjacent the proximal end, an opening adjacent the distal end, and a central lumen communicating therebetween;
a second wire configured for insertion in the blood vessel through the central lumen of the outer wire, the second wire having a proximal end, a distal end, and a generally cylindrical outer surface, wherein the second wire includes an opening adjacent the proximal end, an opening adjacent the distal end, and a central lumen communicating therebetween, and wherein the second wire is longer than the outer wire;
an inner wire configured for insertion in the blood vessel through the central lumen of the second wire, the inner wire having a proximal end, a distal end, and a generally cylindrical outer surface, and wherein the inner wire is longer than the second wire; and
a radio-frequency device including a radio-frequency generating device operatively coupled to one or more of the outer wire, the second wire, and the inner wire, wherein the radio-frequency device is configured to deliver radio-frequency energy to one or more of the distal ends of the outer wire, the second wire, and the inner wire.

2. The multiple-wire system of claim 1, wherein the outer wire has a rigidity greater than a rigidity of the second wire, and the rigidity of the second wire is greater than a rigidity of the inner wire.

3. The multiple-wire system of claim 2, wherein the outer wire, the second wire, and the inner wire are formed substantially of metal alloys, wherein the compositions of the metal alloys are varied to produce the greater rigidity of the outer wire with respect to the second wire and the greater rigidity of the second wire with respect to the inner wire.

4. The multiple-wire system of claim 3, wherein the metal alloys include stainless steel, and further wherein the metal alloy of the outer wire contains a greater proportion of stainless steel as compared to the second wire and the metal alloy of the second wire contains a greater proportion of stainless steel as compared to the inner wire.

5. The multiple-wire system of claim 3, wherein the metal alloys are nitinol alloys.

6. The multiple-wire system of claim 1, wherein the inner wire includes an opening adjacent the proximal end, an opening adjacent the distal end, and a central lumen communicating therebetween.

7. The multiple-wire system of claim 6, wherein the central lumen of the inner wire includes a fluid-impervious coating.

8. The multiple-wire system of claim 1, wherein one or more of the outer wire, the second wire, and the inner wire includes a radio-frequency delivery tip positioned at its distal end, the radio-frequency delivery tip configured to receive radio-frequency energy from the radio-frequency device and deliver the radio-frequency energy to the occlusion in the blood vessel for ablation thereof.

9. The multiple-wire system of claim 1, wherein the radio-frequency device is operatively coupled to the outer wire; and
wherein the outer wire includes a radio-frequency delivery tip positioned at the distal end of the outer wire, the delivery tip configured to receive radio-frequency energy from the radio-frequency device and deliver the radio-frequency energy to the occlusion in the blood vessel for ablation thereof.

10. The multiple-wire system of claim 9, wherein the radio-frequency device is further operatively coupled to the second wire; and
wherein the second wire includes a radio-frequency delivery tip positioned at the distal end of the second wire, the delivery tip configured to receive radio-frequency energy from the radio-frequency device and deliver the radio-frequency energy to the occlusion in the blood vessel for ablation thereof.

11. The multiple-wire system of claim 10,
wherein the radio-frequency device is further operatively coupled to the inner wire; and
wherein the inner wire includes a radio-frequency delivery tip positioned at the distal end of the inner wire, the delivery tip configured to receive radio-frequency energy from the radio-frequency device and deliver the radio-frequency energy to the occlusion in the blood vessel for ablation thereof.

12. The multiple-wire system of claim 1,
wherein the radio-frequency device is operatively coupled to the inner wire; and
wherein the inner wire includes a radio-frequency delivery tip positioned at the distal end of the inner wire, the delivery tip configured to receive radio-frequency energy from the radio-frequency device and deliver the radio-frequency energy to the occlusion in the blood vessel for ablation thereof.

13. The multiple-wire system of claim 12,
wherein the radio-frequency device is further operatively coupled to the second wire; and
wherein the second wire includes a radio-frequency delivery tip positioned at the distal end of the second wire, the delivery tip configured to receive radio-frequency energy from the radio-frequency device and deliver the radio-frequency energy to the occlusion in the blood vessel for ablation thereof.

14. The multiple-wire system of claim 1, wherein one or more of the outer wire, the second wire, and the inner wire have a textured outer surface that aids with the passage of the respective wire through the blood vessel in response to a user manipulating the respective wire.

15. The multiple-wire system of claim 1,
wherein the inner wire has a textured outer surface that aids with the passage of the inner wire through the blood vessel in response to a user manipulating the inner wire; and
wherein the central lumen of the second wire has a textured surface that compliments the textured outer surface of the inner wire, so that when the inner wire is manipulated by a user, the inner wire is displaced through the second wire.

16. The multiple-wire system of claim 1,
wherein the outer wire has a textured outer surface that aids with the passage of the outer wire through the blood vessel in response to a user manipulating the outer wire; and
wherein the second wire has a textured outer surface that aids with the passage of the second wire through the blood vessel in response to a user manipulating the second wire; and
wherein the inner wire has a textured outer surface that aids with the passage of the inner wire through the blood vessel in response to a user manipulating the inner wire.

17. The multiple-wire system of claim 16, wherein the outer wire, the second wire, and the inner wire include a plurality of wound wire strands that define the textured outer surfaces.

18. The multiple-wire system of claim 16, wherein the distal ends of the outer wire, the second wire, and the inner wire each include a cutting edge configured to aid in the passage of the respective wire through the blood vessel.

19. The multiple wire system of claim 1, wherein one or more of the outer wire, the second wire, and the inner wire include a plurality of wound wire strands, the wire strands defining a textured outer surface that aids with the passage of the respective wire through the blood vessel in response to a user manipulating the respective wire about the wire's central axis.

20. The multiple-wire system of claim 1, wherein the inner wire is between about 150 cm and about 300 cm long.

21. The multiple-wire system of claim 20, wherein second wire is between about 5 cm and about 25 cm shorter than the inner wire, and the outer wire is between about 5 cm and about 25 cm shorter than the second wire.

22. A multiple-wire system for percutaneous insertion in a blood vessel, comprising:
an outer wire having a proximal end, a distal end, and a generally cylindrical and textured outer surface, wherein the outer wire includes an opening adjacent the proximal end, an opening adjacent the distal end, and a central lumen communicating therebetween, and wherein the textured outer surface is configured to aid with the passage of the outer wire through the blood vessel in response to a user manipulating the outer wire;
a second wire configured for insertion through the central lumen of the outer wire, the second wire having a proximal end, a distal end, and a generally cylindrical and textured outer surface, wherein the second wire is longer than the outer wire, and wherein the textured outer surface is configured to aid with the passage of the second wire through the blood vessel in response to a user manipulating the second wire; and
a radio-frequency device including a radio-frequency generating device operatively coupled to one or both of the outer wire and the second wire, wherein the radio-frequency device is configured to deliver radio-frequency energy to one or both of the distal ends of the outer wire and the second wire.

23. The multiple-wire system of claim 22, wherein the second wire includes an opening adjacent the proximal end, an opening adjacent the distal end, and a central lumen communicating therebetween.

24. The multiple-wire system of claim 23, wherein the central lumen of the second wire includes a fluid-impervious coating.

25. The multiple-wire system of claim 22, wherein the outer wire has a rigidity greater than a rigidity of the second wire.

26. The multiple-wire system of claim 22, further comprising:
an inner wire configured for insertion through the central lumen of the second wire, the inner wire having a proximal end, a distal end, and a generally cylindrical and textured outer surface, wherein the inner wire is longer than the second wire, and wherein the textured outer surface is configured to aid with the passage of the inner wire through the blood vessel in response to a user manipulating the inner wire.

27. The multiple-wire system of claim 26, wherein the inner wire includes an opening adjacent the proximal end, an opening adjacent the distal end, and a central lumen communicating therebetween.

28. The multiple-wire system of claim 26, wherein the outer wire has a rigidity greater than a rigidity of the second wire and the rigidity of the second wire is greater than a rigidity of the inner wire

29. The multiple-wire system of claim 22, wherein the outer wire and the second wire include a plurality of wound wire strands that define the textured outer surfaces.

30. The multiple-wire system of claim 22,
wherein the distal end of the outer wire includes a cutting edge configured to aid in the passage of the outer wire through the blood vessel; and
wherein the distal end of the second wire includes a cutting edge configured to aid in the passage of the second wire through the blood vessel.

31. The multiple-wire system of claim 22, wherein the central lumen of the outer wire has a textured surface that compliments the textured outer surface of the second wire, so that when the second wire is manipulated by a user, the second wire is displaced through the outer wire.

32. The multiple-wire system of claim 22,
wherein the outer wire includes a plurality of wound wire strands defining the textured outer surface of the outer wire; and
wherein the second wire includes a plurality of wound wire strands defining the textured outer surface of the second wire.

33. A method of ablating an occlusion within a blood vessel, comprising:
percutaneously inserting into a blood vessel an outer wire having a central lumen;
feeding the outer wire through one or more blood vessels;
feeding a second wire having a central lumen through the central lumen of the outer wire;
feeding an inner wire through the central lumen of the second wire to reach the occlusion;
manipulating one or more of the outer wire, the second wire, and the inner wire to engage the occlusion; and
applying radio-frequency energy to a distal end of one or more of the outer wire, the second wire, and the inner wire to ablate the occlusion.

34. The method of claim 33, wherein the outer wire has a rigidity greater than a rigidity of the second wire, and the rigidity of the second wire is greater than a rigidity greater than the inner wire.

35. The method of claim 34, wherein the outer wire, the second wire, and the inner wire are formed substantially of metal alloys, wherein the compositions of the metal alloys are varied to produce the greater rigidity of the outer wire with respect to the second wire and the greater rigidity of the second wire with respect to the inner wire.

36. The method of claim 35, wherein the metal alloys include stainless steel, and further wherein the metal alloy of the outer wire contains a greater proportion of stainless steel as compared to the second wire and the metal alloy of the second wire contains a greater proportion of stainless steel as compared to the inner wire.

37. The method of claim 35, wherein the metal alloys are nitinol alloys.

38. The method of claim 33, wherein the inner wire has a central lumen, the method further comprising:
injecting a fluid through one or more of the central lumens of the outer wire, the second wire, and the inner wire to ablate the occlusion.

39. The method of claim 33, wherein the outer wire, the second wire, and the inner wire each have a textured outer surface that aids with the passage of the respective wire through the one or more blood vessels, the method further comprising.

40. The method of claim 39,
wherein the textured outer surfaces are each defined by a plurality of wound wire strands; and
wherein the manipulating one or more of the outer wire, the second wire, and the inner wire includes twisting the respective wire to at least partially screw into the occlusion.

41. A method of ablating an occlusion within a blood vessel, comprising:
percutaneously inserting into a blood vessel an inner wire;
feeding the inner wire through one or more blood vessels;
feeding a second wire having a central lumen over the inner wire;
feeding an outer wire having a central lumen over the second wire;
manipulating one or more of the inner wire, the second wire, and the outer wire to engage the occlusion; and
applying radio-frequency energy to a distal end of one or more of the inner wire, second wire, and outer wire to ablate the occlusion.

42. The method of claim 41, wherein the outer wire has a rigidity greater than a rigidity of the second wire, and the rigidity of the second wire is greater than a rigidity greater than the inner wire.

43. The method of claim 42, wherein the outer wire, the second wire, and the inner wire are formed substantially of metal alloys, wherein the compositions of the metal alloys are varied to produce the greater rigidity of the outer wire with respect to the second wire and the greater rigidity of the second wire with respect to the inner wire.

44. The method of claim 43, wherein the metal alloys include stainless steel, and further wherein the metal alloy of the outer wire contains a greater proportion of stainless steel as compared to the second wire and the metal alloy of the second wire contains a greater proportion of stainless steel as compared to the inner wire.

45. The method of claim 43, wherein the metal alloys are nitinol alloys.

46. The method of claim 41, wherein the inner wire has a central lumen, the method further comprising:
injecting a fluid through one or more of the central lumens of the outer wire, the second wire, and the inner wire to ablate the occlusion.

47. The method of claim 41, wherein the outer wire, the second wire, and the inner wire each have a textured outer surface that aids with the passage of the respective wire through the one or more blood vessels, the method further comprising.

48. The method of claim 47,
wherein the textured outer surfaces are each defined by a plurality of wound wire strands; and
wherein the manipulating one or more of the outer wire, the second wire, and the inner wire includes twisting the respective wire to at least partially screw into the occlusion.

49. A method of ablating an occlusion within a blood vessel, comprising:
percutaneously inserting into a blood vessel a first wire having a central lumen;
feeding the first wire through one or more blood vessels;
feeding a second wire through the central lumen of the first wire;
feeding a third wire having a central lumen over the first wire;
manipulating one or more of the first wire, the second wire, and the third wire to engage the occlusion; and
applying radio-frequency energy to a distal end of one or more of the first wire, second wire, and third wire to ablate the occlusion.

50. The method of claim 49, wherein the outer wire has a rigidity greater than a rigidity of the second wire, and the rigidity of the second wire is greater than a rigidity greater than the inner wire.

51. The method of claim 50, wherein the outer wire, the second wire, and the inner wire are formed substantially of metal alloys, wherein the compositions of the metal alloys are varied to produce the greater rigidity of the outer wire with respect to the second wire and the greater rigidity of the second wire with respect to the inner wire.

52. The method of claim 51, wherein the metal alloys include stainless steel, and further wherein the metal alloy of the outer wire contains a greater proportion of stainless steel as compared to the second wire and the metal alloy of the second wire contains a greater proportion of stainless steel as compared to the inner wire.

53. The method of claim 51, wherein the metal alloys are nitinol alloys.

54. The method of claim 49, wherein the inner wire has a central lumen, the method further comprising:
injecting a fluid through one or more of the central lumens of the outer wire, the second wire, and the inner wire to ablate the occlusion.

55. The method of claim 49, wherein the outer wire, the second wire, and the inner wire each have a textured outer surface that aids with the passage of the respective wire through the one or more blood vessels, the method further comprising.

56. The method of claim 55,
wherein the textured outer surfaces are each defined by a plurality of wound wire strands; and
wherein the manipulating one or more of the outer wire, the second wire, and the inner wire includes twisting the respective wire to at least partially screw into the occlusion.
